(19) United States
(12) Patent Application Publication
(10) Pub. No.: US 2007/0298090 A1
(43) Pub. Date: Dec. 27, 2007

(54) INTRAORAL DELIVERY OF NICOTINE FOR SMOKING CESSATION

(75) Inventors: Li-Lan H. CHEN, Edison, NJ (US); Alfred LIANG, Edison, NJ (US)

Correspondence Address: LUCAS & MERCANTI, LLP, 475 PARK AVENUE SOUTH, 15TH FLOOR, NEW YORK, NY, 10016, US

(73) Assignee: LAVIPHARM LABORATORIES, INC., East Windsor, NJ (US)

(21) Appl. No.: 11/852,034

(22) Filed: Sep. 7, 2007

Related U.S. Application Data
(63) Continuation of application No. 10/125,696, filed on Apr. 18, 2002.
(60) Provisional application No. 60/285,404, filed on Apr. 20, 2001.

(51) Int. Cl. A61K 9/70; A61K 31/465; A61P 25/34

(52) U.S. Cl. 424/448; 514/343

(57) ABSTRACT

Dosage forms of a nicotine delivery system are disclosed in which a mucoadhesive film, made up of one or more non-microbial hydrocolloid(s) and an effective dose of nicotine, dissolves when applied intraorally to release the nicotine which is absorbed through the oramucosac and directly reaches systemic circulation. Methods for preparing various versions of the dosage forms are disclosed. Methods to assist smoking cessation or provide substitutes for smoking by administrating the dosage form are also provided.

This application is a continuation of U.S. Ser. No. 10/125,696, filed Apr. 8, 2002, which in turn claims the benefit of U.S. Provisional Application No. 60/285,404, filed Apr. 20, 2001; the disclosures of which are incorporated herein by reference as if set forth herein in their entirety.

The present invention is directed to providing a safe and effective means for delivering nicotine to the blood plasma. It can serve as an aid for people trying to stop smoking cigarettes or as a substitute for cigarettes. Specifically, the invention describes the composition of water-soluble, dissolving intraoral film dosage forms and methods for their manufacture and use.

Nicotine is a naturally occurring drug found in tobacco. It can be introduced into the body through many routes, including the smoking of cigarettes. Unfortunately, introducing nicotine into the body in this manner also introduces many other compounds, some of which are deposited onto the lungs and can cause adverse health effects. There is also risk to bystanders in the form of second-hand inhalation of cigarette smoke which has also been shown to cause adverse health effects. Smoking has become increasingly disfavored in recent years and many restrictions have been placed on where an individual may smoke.

The adverse effects of cigarette smoking have inspired many attempts to provide acceptable substitutes for cigarettes and aids to assist individuals to stop smoking. Cessation of the use of tobacco may be followed by withdrawal symptoms that can include cravings, irritability, anxiety, restlessness, headaches, increased appetite, insomnia, drowsiness, difficulty in concentrating, and gastrointestinal complaints. The use of nicotine supplements during the withdrawal period has been shown to provide some relief from the symptoms and to increase the rate of success for those who are trying to quit smoking.

Intraoral delivery provides many advantages. Drugs are absorbed from the oral cavity through the oramucosae, and are transported through the deep lingual or facial vein, internal jugular vein and bracocephalic vein directly into the system circulation. This circumvents the hepatic first-pass effect that can degrade drugs during their transport from initial ingestion to systemic circulation. In addition, the food or gastric emptying rate does not influence the rate of drug absorption. The membranes that line the oral cavity are also easily accessible. As a result, application is painless and precise dosage form localization is possible. The oral cavity is routinely exposed to a multitude of foreign compounds and physical injuries, and so has evolved into a robust membrane that is less prone to irreversible damage by the drug or dosage form.

The local environment at the selected site of administration can be easily controlled by, for example, modifying pH and ionic composition of the dose. Co-administration of permeability enhancers or protease inhibitors will modify absorption in a well-defined area. Intraoral administration may be preferred, for example, for “nil-by-mouth” patients, if either nausea or vomiting is a problem, if the subject is unconscious, in subjects with upper gastrointestinal tract disease or surgery which affects gastric absorption, or in subjects who have difficult swallowing peroral medications.

Dissolving films have been mentioned as dosage forms in previous disclosures. PCT Patent Application WO 00/18365 described a consumable film that was dependant on pullulan, a microbial hydrocolloid. U.S. Pat. Nos. 5,629,003 and 5,948,430 discussed dissolving films for general uses, some including the delivery of drugs, but were not formulated for rapid absorption through the oramucosae.

Various delivery systems have been used for the intraoral delivery of nicotine, including gum, capsules, tablets, and lozenges. (U.S. Pat. No. 5,662,920 and U.S. Pat. No. 4,806,356 related to nicotine lozenges; PCT Patent Application WO 88/03803 related to chewable nicotine capsules; Belgian Patent BE 899037 related to nicotine tablets; and U.S. Pat. No. 5,783,207 related to nicotine-containing lollipops). None of these substitutes can fully meet the needs of the smoker. These devices release their nicotine slowly, providing a low, constant level of nicotine in the blood plasma. This may relieve some symptoms of nicotine withdrawal, but it will not simulate the effect of smoking a cigarette, which causes a quick peak of nicotine in the blood plasma and gradually fades away.

Nicotine nasal spray was found to provide a profile close to that of a cigarette due to the nasal membrane's high permeability. However, delivery of nicotine through the nose can irritate the nose and cause various adverse effects, such as watery eyes, runny nose, coughing, sneezing and nasal ulcers.

To date, it has been difficult to deliver nicotine into the system in a way that both results in blood plasma levels that mimic those achieved by consistent smoking and is comfortable and safe to use.

### SUMMARY OF THE INVENTION

The present invention describes a novel nicotine delivery system. The dosage form is a monolithic or bilayer mucoadhesive film, which is made up of one or more non-microbial hydrocolloid(s) and an effective dose of nicotine in either the neutral or charged state. The mucoadhesive film dissolves when applied intraorally to release the nicotine which is absorbed through the oramucosac and directly reaches systemic circulation. In addition to nicotine and non-microbial hydrocolloid(s), the delivery system may further include one or more emulsifiers, release modifiers, taste modifying agents, plasticizers, water soluble inert fillers, preservatives, buffering agents, stabilizers or coloring agents.

The present invention also describes various methods for making the dosage form by mixing the nicotine, in either neutral or charged form, with the non-microbial hydrocolloid(s) and any emulsifiers, release modifiers, taste modifying agents, plasticizers, water soluble inert fillers, preservatives, buffering agents, stabilizers or coloring agents in an aqueous and/or alcoholic solution and forming a homogenous coating solution or speadable mass. The homogenous coating solution is degassed completely and uniformly coated onto a casting liner with a predetermined thickness. The cast film could be a homogenous monolayer or bilayer, in which one layer contains ionized nicotine and the other layer contains buffering agents to convert nicotine from an ionized state to a neutral state upon dissolution. Alternatively, a spreadable mass can be made and is extruded to form a film on a casting liner through a twin-screw extruder. The extruded film is then dried. The dried film from the cast or extrusion is die-cut into various sizes of dosage units. The dissolution of the resulted films can be programmed and controlled during manufacture.

The present invention also includes methods of assisting cessation of smoking or providing a substitute for smoking consisting of administering one of the dosage forms described above.

### GLOSSARY

“Active agents” include nicotine base and its salts. Nicotine salts include any physiologically acceptable salts, such as hydrochloride, dihydrochloride, sulfate, tartrate, ditartrate, zinc chloride, salicylate, alginate, ascorbate, benzoate, citrate, edetate, fumarate, lactate, maleate, oleate and sorbate, formed by the interaction of nicotine and any acid.

“Buffering agents” include acidulants and alkalizing agents exemplified by citric acid, fumaric acid, lactic acid, tartaric acid, malic acid, as well as sodium citrate, sodium bicarbonate and carbonate, and sodium or potassium phosphate.

“Coating solution” is a viscous and homogeneous mixture of hydrocolloids, nicotine and other additives in an aqueous solution.

“Coloring agents” can include FD & C coloring agents, natural coloring agents, and natural juice concentrates, pigments such as titanium oxide, silicon dioxide and zinc oxide.

“Disintegration time” is the time (in seconds) at which a film breaks when brought into contact with water or saliva. In an embodiment of the invention, the disintegration time ranges from 1-600 seconds, more preferably 10-300 seconds.

“Dissolving time” is the time (seconds or minutes) at which not less than 80% of the tested film is dissolved in an aqueous media or saliva. In an embodiment of the invention, the dissolving time ranges from 0.1-120 minutes with a preferred range of 0.5-60 minutes.

“Effective dose of nicotine” is the amount of nicotine required to result in the desired level of nicotine in a subject's blood plasma.

“Emulsifying agents” include solubilizers, wetting agents and releasing modifiers and are exemplified by polyvinyl alcohol, sorbitan ester, benzyl benzoate, glyceryl monostearate, polyoxyethylene alkyl ethers, polyoxyethylene stearates, polyoxamer, polyoxyethylene castor oil derivatives, hydrogenated vegetable oils, bile salts, tween, span and ethanol.

“Enzyme inhibitor” is a natural or synthetic molecule which inhibits enzymatic metabolism of an active agent in the saliva or in a mucosal tissue.

“The hydration rate” is the speed of absorbing water at 25° C. and 75% relative humidity in 24 hours.

“Percentage of swelling” is the percentage of the initial volume that is increased before dissolving.

“Permeation enhancer” is a natural or synthetic molecule which facilitates the absorption of an active agent through a mucosal surface.

“Plasticizers” can include glycerin, sorbitol, propylene glycol, polyethylene glycol, triacetin, triethyl citrate (TEC), acetyl triethyl citrate (ATBC) and other citrate esters.

“Preservatives” include anti-microbial agents and non-organic compounds and are exemplified by sodium benzoate, parabens and derivatives, sorbic acid and its salts, propionic acid and its salts, sulfur dioxide and sulfites, acetic acid and acetates, nitrites and nitrates.

“Release study” is the percentage of drugs released from the film as a function of time in a suitable dissolution vessel and medium under specified conditions of temperature and pH.

“Stabilizers” include anti-oxidants, chelating agents, and enzyme inhibitors as exemplified by ascorbic acid, vitamin E, butylated hyroxyanisole (BHA), butylated hydroxytoluene (BHT), propyl gallate, dilauryl thiodipropionate, thiodipropionic acid, gum guaiac, citric acid, edetic acid and its salts and glutathione.

“Subject” is a human or animal species.

“Taste modifying agents” include flavoring agents, sweetening agents and taste masking agents and are exemplified by; the essential oils or water soluble extracts of menthol, wintergreen, peppermint, sweet mint, spearmint, vanillin, cherry, butterscotch, chocolate, cinnamon, clove, lemon, orange, raspberry, rose, spice, violet, herbal, fruit, strawberry, grape, pineapple, peach, kiwi, papaya, mango, coconut, apple, coffee, plum, watermelon, nuts, durean, green tea, grapefruit, banana, butter, chamomile, sugar, dextrose, lactose, mannitol, sucrose, xylitol, malitol, acesulfame potassium, aspartame, saccharin, sodium saccharin, sodium cyclamate and honey.

“Thickness” is determined by measurement in mil (a mil=one thousandth of an inch) when a film is placed between two microscopic slides.

“Water Content” is defined here and in the claims as % residual water content per unit dose as measured according to the Karl Fisher method and expressed as percent of the dry weight of the film.

“Water soluble inert fillers” include mannitol, xylitol, sucrose, lactose, maltodextrin, dextran, dextrin, modified starches, dextrose, sorbitol, and dextrates.

### BRIEF DESCRIPTION OF THE DRAWINGS

There are shown in the drawings certain exemplary embodiments of the present invention as presently preferred. It should be understood that the present invention is not limited to the embodiments disclosed as examples, and is capable of variation within the spirit and scope of the appended claims.

In the drawings,

FIG. 1 is an illustration of a monolayer nicotine-containing intraoral film;

FIG. 2 is an illustration of a bilayer nicotine-containing intraoral film;

FIG. 3 is a graphical representation of nicotine dissolution profiles as a function of film thickness;

FIG. 4 is a graphical representation comparing dissolution profiles among various formulations of nicotine-containing films;

FIG. 5 is a graphical representation of the amount of nicotine released over time from a bilayer intraoral film;

FIG. 6 is a graphical representation comparing nicotine plasma levels over time from three sources: nicotine-containing intraoral film, nicotine chewing gum, and a nicotine inhaler;

FIG. 7 is a graphical representation comparing nicotine plasma levels over time from three sources: nicotine-containing intraoral film, nicotine nasal spray, and smoking a cigarette;

FIG. 8 is a graphical representation of the fitted and observed plasma concentrations of nicotine over time after using nicotine therapeutic delivery systems in the forms of intraoral film, inhaler, and gum;

FIG. 9 is a graphical representation of predicted nicotine levels in the oral cavity over time for various nicotine delivery rates;

FIG. 10 is a graphical representation of nicotine concentration in the oral cavity over time with predicted plasma level compared with observed nicotine plasma level for a nicotine-containing intraoral film of the present invention; and,

FIG. 11 is a graphical representation of nicotine concentration in the oral cavity over time with predicted plasma level compared with observed nicotine plasma level from nicotine-containing chewing gum (2 mg).

### DETAILED DESCRIPTION

The present invention is related to compositions and methods of manufacture which facilitate the intraoral delivery of nicotine to an individual so that the nicotine quickly and directly enters the individual's systemic circulation. The dosage form, a thin nicotine-containing film, permits intraoral delivery of nicotine through the oramucosae of the mouth, pharynx and esophagus. This dosage form is capable of delivering nicotine into a subject's blood in a pattern which is similar to smoking a cigarette. In addition, the film, provides the subject an unobtrusive and unnoticeable method to relieve cigarette craving and aid in smoking cessation which has greater social acceptability and patient compliance than previous forms of nicotine substitutes.

The present invention overcomes several of the limitations associated with other nicotine delivery systems, such as chewing gum, transdermal patches, nasal sprays, inhalers, sublingual tablets, lozenges and lollipops. There are four primary advantages of the present invention. The first advantage is the capability of programming the release of nicotine in a controlled manner during manufacture. The second advantage is the capability of releasing highly permeable nicotine base at administration, though stored in ionized form to eliminate the loss during manufacture and improve nicotine stability. The third advantage is the capability of rapid absorption through the oramucosae to achieve fast onset of action and quickly relieve subjects' cravings. The fourth advantage is the unobtrusive and unnoticeable administration which can lead to greater patient compliance and, better social acceptability.

The dosage form is a monolithic or bilayer mucoadhesive film, which is made up of one or more non-microbial hydrocolloid(s), and an effective dose of nicotine in either the neutral or charged state. The film dissolves when applied intraorally to release the nicotine which is absorbed through the oramucosae and directly reaches systemic circulation. In addition to nicotine and non-microbial hydrocolloid(s), the delivery system may further include one or more emulsifiers, release modifiers, taste modifying agents, plasticizers, water soluble inert fillers, preservatives, buffering agents, stabilizers or coloring agents.

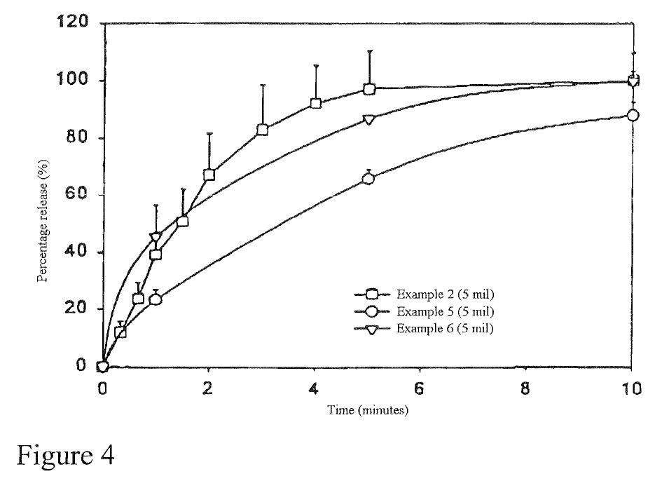

In a preferred embodiment, the dosage form is an intraoral quick-dissolving film which is applied lingually. The dosage form is applied to the tongue and adheres to the palate as soon as a subject closes his or her mouth. Then, the film rapidly disintegrates, dissolves and releases highly permeable nicotine base for oramucosal absorption. The release of nicotine occurs without mastication, such as holding, chewing or sucking of the dosage form. Subjects do not need to stop or alter their activities in any way. There is almost no risk that a subject will choke or accidentally swallow the whole dosage form, which may occur with tablets, capsules or lozenges. The dosage form in the present invention does not interrupt a subject's speech pattern. The dissolution of the films can be programmed and controlled during manufacture as shown in FIGS. 3 and 4.

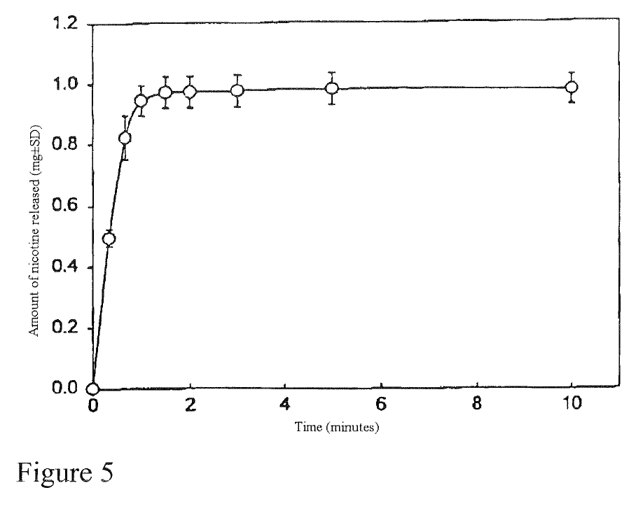

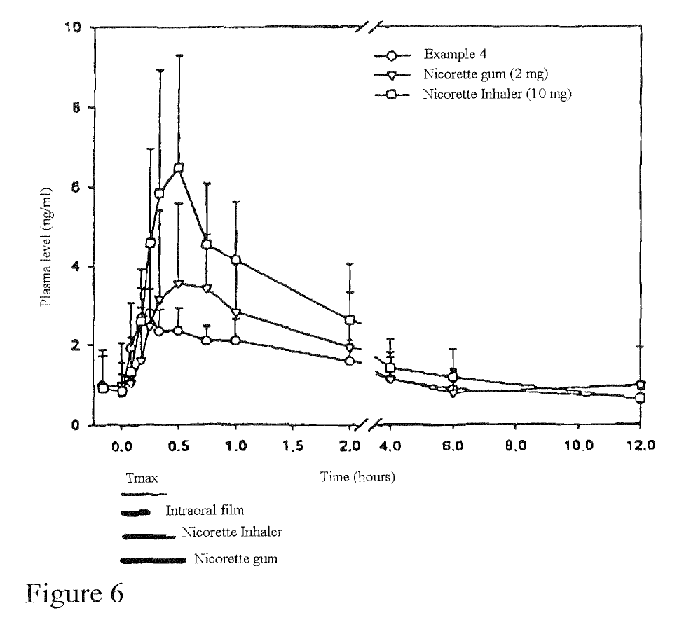

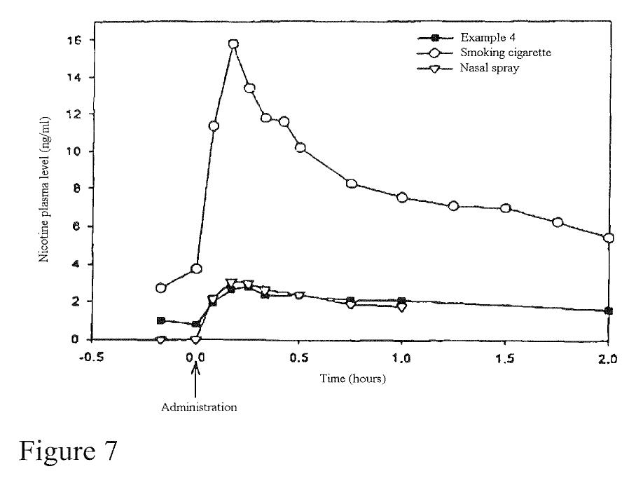

With the rapidly dissolving nicotine-containing dosage form in the present invention (FIG. 5), the released nicotine is rapidly absorbed by the oramucosae and quickly reaches systemic circulation. The time to nicotine peak is within 15 minutes, as shown in FIG. 6. Therefore, it is possible to achieve a relatively rapid initial increase in blood nicotine concentration followed by a maintenance period of lower blood nicotine concentration and thereby simulate the pattern obtained by smoking a cigarette or taking a nasal spray, as shown in FIG. 7. This self-administered, convenient, and unobtrusive nicotine therapeutic delivery system provides the real and perceived value of instant relief for nicotine withdrawal symptoms.

The properties of the nicotine-containing quick-dissolving film are substantially determined by the viscosity of the hydrocolloid(s) it contains, which is further dependent on molecular size, derivation, charge, hydrophobicity and hydrophilicity and the presence of other additives in the formulation. According to embodiments of the invention, when synthetic hydrocolloids are selected, a high concentration of lower viscosity polymers is preferred. In embodiments of the invention, a hydrocolloid concentration in the range of 50-90% of the dry weight of the filins is provided, more particularly greater than 60%. Films with hydrocolloidal content in this range have dry tack and wet tack properties that improve ease of handling and use. The low dry tack properties of the film provide for a physically attractive and easily handled film that is neither fragile nor sticky and can be easily removed from packaging and placed on a mucosal surface. The wet tack properties of the film provide the advantage of stickiness in the moistened film so that when the film is placed on the oramucosac, it remains attached until it dissolves. In contrast, if the wet tack is too low, the film could move in the mouth and may be swallowed before dissolving and possibly give rise to choking. Furthermore, the low moisture content and low dry tack of the film enhances the shelf-life of the film and the flexibility of the dosage forms. These properties render the films suitable for easy manufacturing, packaging, handling and application.

In a preferred embodiment of the invention, a water-soluble polymer is selected having a gelation temperature greater than 70° C. and providing quick disintegration and rapid dissolution. The hydration rate of a hydrocolloid having these features is rapid with a percentage moisture absorption of polymers in the range of 5-20% at 75% humidity at room temperature. The hydration rate is selected according to the desired wettability of the film thereby obviating the need for surfactants. The wet tack of the hydrated film ranges from 35-150 grams, more particularly 40-100 grams. The percentage swelling may be less than 10% within 60 seconds.

In a preferred embodiment, the film is cast so as to have a reduced thickness for enhanced flexibility where the thickness of the film is 1-50 mil, more preferably 2-40 mil, as illustrated in FIG. 3. The water content of the film ranges from 0.5-10% with a preferred range of 1-5%.

In an embodiment of the invention, a film may be formed using a mixture of two or more types of the same hydrocolloid that differ only in molecular weights and/or different degrees of substitution. In addition, anionic polymers can be added into the formulation to modify the nicotine dissolution profile (FIG. 4). The time of dissolution of the film is in the range of 30 seconds to 60 minutes; the time of disintegration of the film may be 1-600 seconds, preferably 10-300 seconds.

In embodiments of the invention, the hydrocolloid(s) may be water-soluble and non-gelling (at room temperature) natural gum or derivatives, including pectin and derivatives, gum arabic, tragacanth gum, alginate and derivatives, modified starches, gum ghatti, okra gum, karaya gum, dextrins and maltodextrins, konjac, acemannan from aloe, locust bean gum, tara gum, quince seed gum, fenugreek seed gum, psyllium seed gum, tamarind gum, oat gum, quince seed gum, carrageenans, larch arabinogalactan, flaxseed gum, chondroitin sulfates, hyaluronic acid, chitosan, and rhizobium gum.

In embodiments of the invention, the hydrocolloid(s) may be water-soluble and non-gelling polypeptides or proteins exemplified by gelatin, albumins, milk proteins, soy protein, and whey proteins.

In embodiments of the invention, the hydrocolloid(s) may be water-soluble synthetic polysaccharides exemplified by any of the following: polyethylene-imine, hydroxyethyl cellulose, sodium carboxymethyl cellulose, carboxymethyl cellulose, hydroxypropyl cellulose, hydroxypropyl methylcellulose, methyl cellulose, ethyl cellulose, polyacrylic acids, polyacrylamides, carbopols, polyvinylpyrrolidone, polyethylene glycols, polyethylene oxides and polyvinyl alcohols.

A preferred embodiment of the invention utilizes a hydroxypropyl methyl cellulose having a methoxy content of about 19-30% and hydroxypropyl content of 7-12% and a molecular weight of approximately 50,000-250,000 daltons.

In addition to hydrocolloid(s) and nicotine, the film may contain any or all of the following ingredients: emulsifiers, release modifiers, taste modifying agents, plasticizers, water soluble inert fillers, preservatives, buffering agents, stabilizers or coloring agents.

In a preferred embodiment of the invention, the percentage dry weight concentration of at least single ingredients incorporated into a film in each of the following categories is as follows: emulsifying agent (0.01%-5%), plasticizer (0.5-20%), nicotine (0.01-20%), taste modifying agents (0.1-20%), coloring agents (0.01-2%), water soluble inert fillers (0.5-50%), preservatives (0.01-5%), buffering agents (0.01-20%) and stabilizers (0.01-5%).

Administration of a dosage form described above to an individual who wants to stop smoking is a method for assisting smoking cessation. Administration of a dosage form described above to an individual is a method for providing a substitute for smoking.

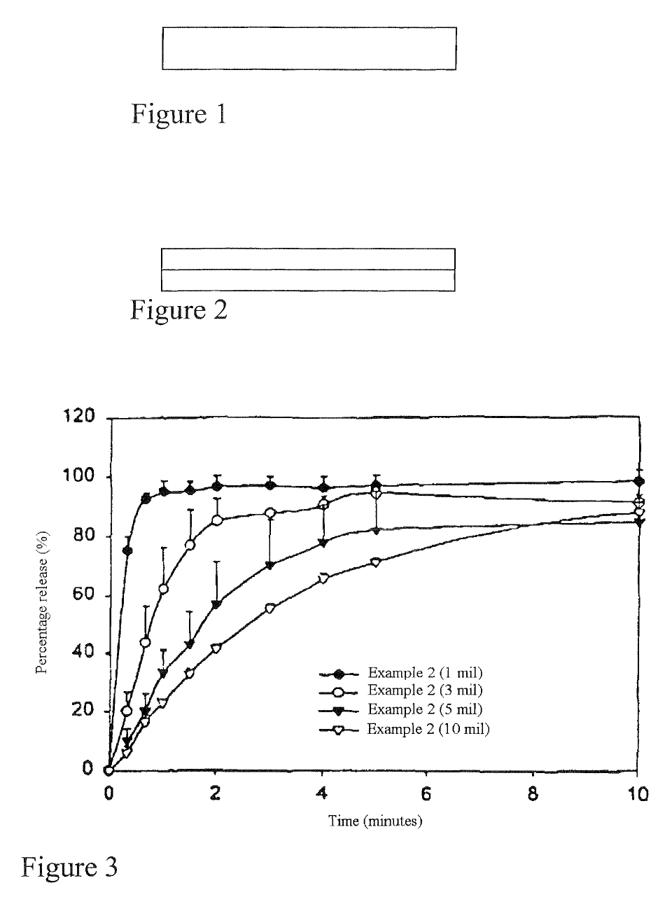

A method for making the dosage form involves mixing the nicotine, in either neutral or charged fonn with the non-microbial hydrocolloid(s) and any emulsifiers, release modifiers, taste modifying agents, plasticizers, water soluble inert fillers, preservatives, buffering agents, stabilizers or coloring agents in an aqueous and/or alcoholic solution and forming a homogenous coating solution or speadable mass. The homogenous coating solution is degassed completely and uniformly coated onto a casting liner with predetermined thickness. The cast film is subsequently dried. The cast film can be a homogenous monolayer (FIG. 1) or bilayer (FIG. 2), in which one layer contains ionized nicotine and the other layer contains buffering agents to convert nicotine from an ionized state to a neutral state upon dissolution. Alternatively, a spreadable mass can be made and is extruded to form a film on a casting liner through a twin-screw extruder. The dried film from the cast or extrusion is die-cut into various sizes of dosage units and can be pouched into Barex pouching material and/or Teflon-like blisters, such as Aclar.

The preferred process of manufacturing the monolayer quick-dissolving dosage form of the invention includes the solvent casting method. A natural or synthetic non-microbial hydrocolloid is completely dissolved or dispersed in water or in a water alcoholic solution under mixing to form a homogenous formulation. In addition to nicotine and the non-microbial hydrocolloid, any emulsifiers, release modifiers, taste modifying agents, plasticizers, water soluble inert fillers, preservatives, buffering agents, stabilizers or coloring agents may be added and dispersed or dissolved uniformly in the hydrocolloid solution. This homogeneous nicotine mixture (coating solution) with a solid content of 5-40% and a viscosity of 500-15000 cps is degassed and coated on the non-siliconized side of a polyester film at 2-50 mil wet film thickness and dried under aeration at a temperature between 40-100° C. so as to avoid destabilizing the agents contained within the formulation. The dry film formed by this process is a glossy, substantially transparent, stand-alone, self-supporting, non-tacky and flexible film. The dry film is then cut into a suitable shape and surface area for intraoral administration. The cutting can be accomplished by using a rotary die. The size of the film can be varied according to the dosage required. Films can then be packaged into a single Barex pouch package or multi-unit Aclar blister card.

An alternative process for making a monolayer quick-dissolving dosage form can be used. A spreadable mass is formed from the hydrocolloid(s), nicotine and any emulsifiers, release modifiers, taste modifying agents, plasticizers, water soluble inert fillers, preservatives, buffering agents, stabilizers or coloring agents. It is then deposited into an extrudable mass feeder which leads to a twin screw extruder. The extruded film is deposited onto a casting liner. The film is dried and cut into a suitable shape and surface area for intraoral administration.

The preferred process of making a bilayer nicotine intraoral film is described below. This process allows separation of the alkalizing agents, which are used to modify pH in the oral cavity, from the nicotine salts-containing layer. In such a design, the loss of nicotine during manufacture and storage is eliminated. This process requires that a nicotine coating solution and a non-nicotine coating solution be prepared separately. Acidifying agents are used to reduce the pH of the nicotine coating solution while nicotine base is used. Otherwise, nicotine salts, such as hydrochloride, dihydrochloride, sulfate, tartrate, ditartrate, zinc chloride, salicylate, alginate, ascorbate, benzoate, citrate, edetate, fumarate, lactate, maleate, oleate or sorbate, are used. Alkalizing agents are added into the non-nicotine coating solution. The nicotine and non-nicotine coating solutions are obtained and degassed. The nicotine coating solution is cast and dried at a first station; then, the non-nicotine coating solution is cast and dried on the top of the nicotine layer at a second station. The bilayer film is still a glossy, substantially transparent, stand-alone, self-supporting, non-tacky and flexible film. The dry film is then cut into a suitable shape and surface area for nicotine intraoral administration. The film contains ionized nicotine; however, it is capable of releasing highly permeable nicotine base upon dissolution to provide rapid absorption into systemic circulation. The film exhibits excellent dissolution stability and no apparent nicotine loss as determined by an accelerated stability study the results of which are provided in Table 1.

TABLE 1
Nicotine dissolution stability from double layer intraoral film

| | | Month 1 | | Month 2 | |
|---|---|---|---|---|---|
| | Month 0 | 25° C. | 40° C. | 25° C. | 40° C. |
| % (20 secs) | 55.12 ± 18.01 | 49.98 ± 3.24 | 49.47 ± 2.85 | 57.66 ± 10.95 | 56.67 ± 3.25 |
| % (60 secs) | 92.55 ± 3.63 | 95.04 ± 4.75 | 94.56 ± 5.09 | 95.28 ± 1.67 | 92.01 ± 4.53 |

A film produced using a method detailed above is capable of delivering an effective dose of nicotine when it is administered to the subject by placing it oil a mucosal surface such as the tongue. There it will rapidly dissolve in the saliva (within 0.5-60 minutes) to release nicotine for intraoral absorption. The thin film is simply applied on top of subject's tongue. The dosage form adheres to the site of application immediately. The film disintegrates, dissolves and releases nicotine for rapid intraoral absorption.

The dissolution of the nicotine-containing film can be programmed and controlled in different ways. FIG. 3 shows the release profile of monolayer films with various thicknesses. By increasing film thickness. The dissolution rate can be reduced. FIG. 4 compares the dissolution profiles of various formulations. Using a hydrocolloid with a higher molecular weight, or incorporating an anionic polymer slows down the dissolution of nicotine. FIG. 5 shows the rapid dissolution profile of a bilayer formulation.

In an embodiment of the invention, the film thickness of a monolayer film is adjusted so that nicotine is sustain-released in the range of 1-120 minutes.

In a preferred embodiment of the invention, the film thickness of a bilayer film is adjusted so that nicotine maximum level in the plasma is achieved within 15 minutes.

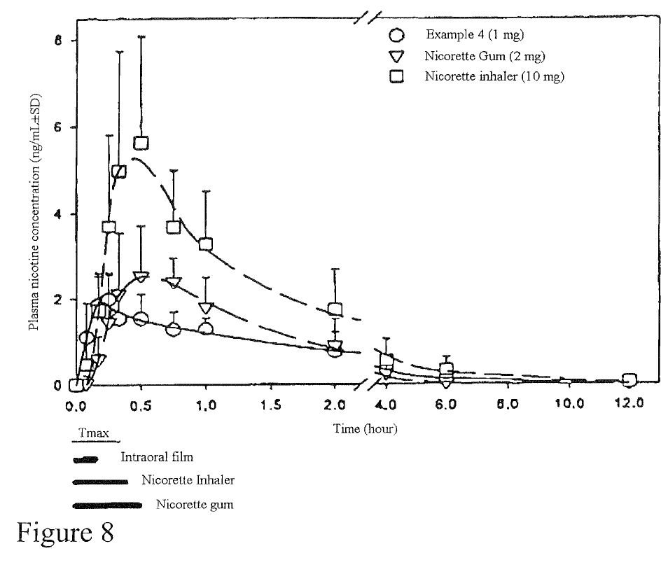

The quick-dissolving nicotine-containing film in the present invention allows rapid release of nicotine for fast intraoral absorption. FIG. 6 compares the plasma level of nicotine delivered via a commercially-available, nicotine-containing chewing gum (Nicorette® gum), a commercially-available, nicotine-containing inhaler (Nicorette® inhaler) or a nicotine-containing intraoral film of the present invention to human subjects. All three products are designed to deliver nicotine into systemic circulation through the oramucosae. Tₘₐₓ for the intraoral film was significantly shorter than the Tₘₐₓ for either Nicorette® gum or an inhaler, and was found to be comparable to nasal spray and smoking a cigarette as shown in FIG. 7. The faster absorption into systemic circulation from the intraoral film in the present invention provides more rapid relief from cigarette craving. The plasma levels were further fitted to a two-compartment pharmacokinetic model and are shown in FIG. 8 and the primary and secondary parameters obtained are tabulated in Tables 2 and 3. The absorption rate constant (Kₐ) obtained from the quick-dissolving film was significantly higher than the gum and inhaler, and the lag time was also significantly shorter for the quick-dissolving film than the gum or inhaler. This corresponds to the shortest Tₘₐₓ observed for the quick-dissolving film. Comparing the quick-dissolving film and Nicorette® gum, though the Cₘₐₓ of film was slightly lower than gum, the AUCs were not significantly different.

TABLE 2
Comparison of the primary pharmacokinetic parameters from modeling

| | Film | Nicorette Gum | Nicorette inhaler |
|---|---|---|---|
| V/F | 290.49 | 193.05 | 322.08 |
| Kₐ, hr⁻¹ | 13.49 | 3.65 | 4.23 |
| Kₑ, hr⁻¹ | 0.82 | 1.23 | 1.30 |
| K₁₂, hr⁻¹ | 6.02 | 0.99 | 2.10 |
| K₂₁, hr⁻¹ | 6.98 | 2.24 | 1.46 |
| Lag time, hr | 0.05 | 0.14 | 0.13 |

TABLE 3
Comparison of the secondary pharmacokinetic
parameters from modeling

| | Film | Nicorette Gum | Nicorette inhaler |
|---|---|---|---|
| Cₘₐₓ (ng/mL) | 1.91 | 2.53 | 5.32 |
| Tₘₐₓ (hr) | 0.18 | 0.53 | 0.43 |
| AUC (ng · hr/mL) | 4.22 | 4.20 | 9.52 |

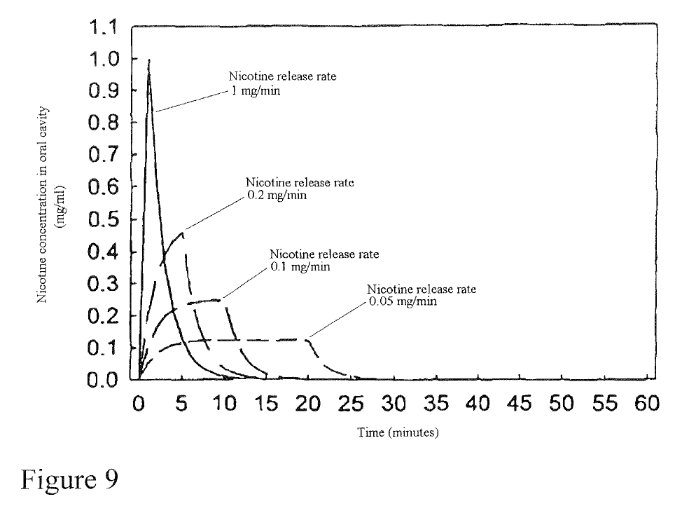

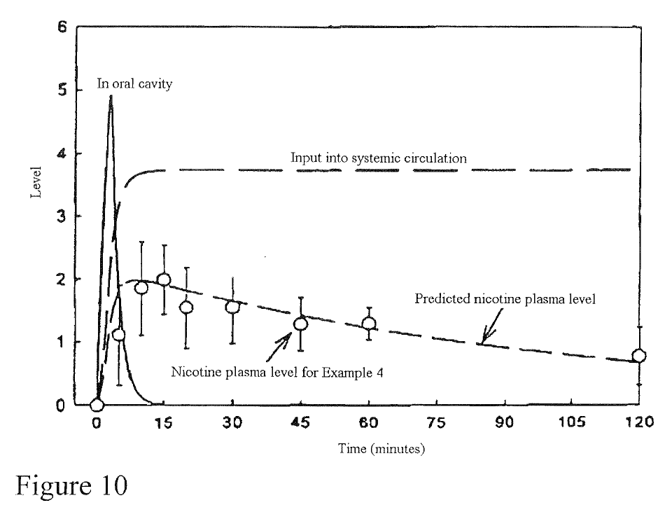

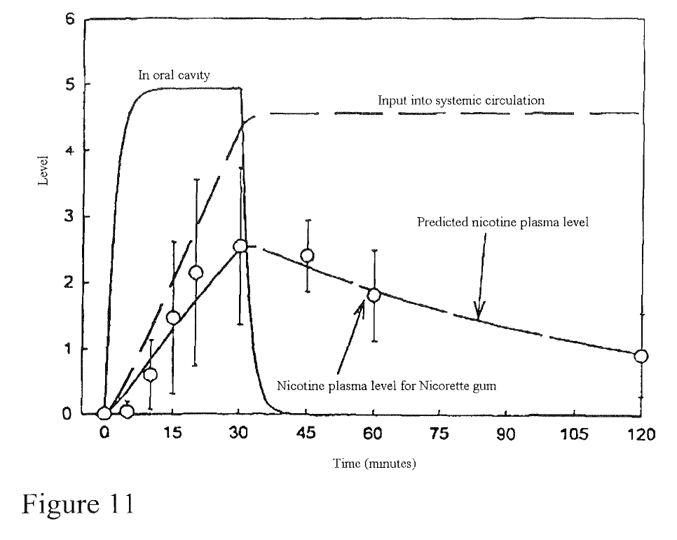

The rate of nicotine absorption via oramucosac and the subsequent plasma profile depend highly on the release patterns from nicotine-containing intraoral delivery systems. The predicted nicotine concentrations in the oral cavity for different nicotine delivery systems with various release rates are illustrated in FIG. 9. The nicotine concentrations are corrected for salivary flow and loss due to swallowing. This predicted nicotine concentration in the oral cavity could be used to calculate the resultant plasma nicotine level, as shown in FIGS. 10 and 11. The symbols (white open circles) in FIGS. 10 and 11 are clinical data obtained from Example 4, whose release rate ranges from 0.5 to 1 mg/min, and Nicorette® gum for which the release rate of 0.033 mg/min was assumed. The predicted and actual plasma levels show good correlation.

### EXAMPLES

The preferred embodiments of the present invention will now be further described through the following examples set forth hereinbelow which are intended to be illustrative of the preferred embodiments of the present invention and are not intended to limit the scope of the invention as set forth in the appended claims.

### Example 1

### Intraoral Monolayer Film Which Contains Ionized Nicotine

0.1 grams of sodium EDTA (stabilizer), 1.5 grams of monoammonium glycyrrhizin (MagnaSweet 100) (sweetener), 0.02 grams of methylparaben/propylparaben 4:1 mix (Nipagin M/Nipasol M) (preservative), 0.5 grams of citric acid (acidifying agent), 0.005 grams of FD&C Red 40, 0.001 grams of Blue 1 and 0.005 grams of Yellow 5 (coloring agents) were completely dissolved in 58.57 grams of water. 20 grams of hydroxypropyl methylcellulose (Methocel E5) (water-soluble film former) was wetted and uniformly mixed with 15 grams of ethanol (wetting agent), 1 gram of butterscotch (flavor), 1.5 grams of propylene glycol (plasticizer), and 1 gram of peppermint oil (flavor). Then the aqueous solution was gradually poured into the wetted Methocel E5 under agitation. After a homogenous viscous solution was obtained, 0.8 grams of nicotine base was added into and mixed with the solution in a well-vented environment. The final coating solution was degassed, cast at 12 mil, dried at 55° C. for 8 minutes and die-cut. The unit dose is shown in FIG. 1.

### Example 2

### Intraoral Monolayer Film Which Contains Neutral and Ionized Nicotine

0.1 grams of sodium EDTA (stabilizer), 1.5 grams of monoammonium glycyrrhizin (MagnaSweet 100) (sweetener), 0.02 grams of methylparaben4propylparaben 4:1 mix (Nipagin M/Nipasol M) (preservative), 0.005 grams of FD&C Red 40, 0.001 grams of Blue 1 and 0.005 grams of Yellow 5 (coloring agents) were completely dissolved in 59.07 grams of water. 20 grams of hydroxypropyl methylcellulose (Methocel ES) (water-soluble film former) was wetted and uniformly mixed with 15 grams of ethanol (wetting agent), 1 gram of butterscotch (flavor), 1.5 grams of propylene glycol (plasticizer), and 1 gram of peppermint oil (flavor). Then the aqueous solution was gradually poured into the wetted Methocel E5 under agitation. After a homogenous viscous solution was obtained, 0.8 grams of nicotine base was added into and mixed with the solution in a well-vented environment. The final coating solution was degassed, cast at 12 mil, dried at 55° C. for 8 minutes and die-cut. The unit dose is shown in FIG. 1.

### Example 3

### Intraoral Monolayer Film Which Contains Nicotine Base

0.1 grams of sodium EDTA (stabilizer), 1.5 grams of monoammonium glycyrrhizin (MagnaSweet 100) (sweetener), 0.02 grams of methylparaben/propyl/paraben 4:1 mix (Nipagin M/Nipasol M) (preservative), 0.5 grams of sodium bicarbonate (alkalizing agent), 0.005 grams of FD&C Red 40, 0.001 grams of Blue 1 and 0.005 grams of Yellow 5 (coloring agents) were completely dissolved in 57.57 grams of water. 20 grams of hydroxypropyl methylcellulose (Methocel ES) (water-soluble film former) was wetted and uniformly mixed with 15 grams of ethanol (wetting agent), 1.5 grams of butterscotch (flavor), 1.5 grams of propylene glycol (plasticizer), and 1.5 grams of peppermint oil (flavor). Then the aqueous solution was gradually poured into the wetted Methocel E5 under agitation. After a homogenous viscous solution was obtained, 0.8 grams of nicotine base was added into and mixed with the solution in a well-vented environment. The final coating solution was degassed, cast at 12 mil, dried at 55° C. for 8 minutes and die-cut. The unit dose is shown in FIG. 1.

### Example 4

### Intraoral Bilayer Film Containing Ionized Nicotine Converts to Base Upon Dissolution

This process required making a nicotine coating solution and a non-nicotine coating solution. To make the nicotine coating solution, 0.1 grams of sodium EDTA (stabilizer), 1.5 grams of monoammonium glycyrrhizin (MagnaSweet 100) (sweetener), 0.02 grams of methylparaben/propylparaben 4:1 mix (Nipagin M/Nipasol M) (preservative), 0.5 grams of citric acid (acidifying agent), 0.005 grams of FD&C Red 40, 0.001 grams of Blue 1 I and 0.005 grams of Yellow 5 (coloring agents) were completely dissolved in 57.37 grams of water. 20 grams of hydroxypropyl methylcellulose (Methocel E5) (water-soluble film former) was wetted and uniformly mixed with 15 grams of ethanol (wetting agent), 1.2 grams of butterscotch (flavor), 1.5 grams of propylene glycol (plasticizer), and 1.2 grams of peppermint oil (flavor). Then the aqueous solution was gradually poured into the wetted Methocel E5 under agitation. After a homogenous viscous solution was obtained, 1.6 grams of nicotine base was added into and mixed with the solution in a well-vented environment. This nicotine-containing solution was used to manufacture the first layer, and was degassed, cast at 6 mil and dried at 55° C. for 8 minutes.

To make the nicotine-free solution, 0.1 grams of sodium EDTA (stabilizer), 1.5 grams of monoammonium glycyrrhizin (MagnaSweet 100) (sweetener), 0.02 grams of methylparaben/propylparaben 4:1 mix (Nipagin M/Nipasol M) (preservative), 1 gram of sodium bicarbonate (alkalizing agent), 0.005 grams of FD&C Red 40, 0.001 grams of Blue 1 and 0.005 grams of Yellow 5 (coloring agents) were completely dissolved in 58.77 grams of water. 20 grams of hydroxypropyl methylcellulose (Methocel E5) (water-soluble film former) was wetted and uniformly mixed with 15 grams of ethanol (wetting agent), 1.2 grams of butterscotch (flavor), 1.2 grams of propylene glycol (plasticizer), and 1.2 grams of peppermint oil (flavor). Then the aqueous solution was gradually poured into the wetted Methocel E5 under agitation. This nicotine-free solution was cast onto the first layer at 4 mil and dried at 55° C. for another 8 minutes. The schematic illustration of the dosage form is shown in FIG. 2.

### Example 5

### Intraoral Monolayer Film Containing Neutral and Ionized Nicotine

0.1 grams of lauroyl macrogol-32 glycerides (Gelucire 44/14) (release modifier) was dissolved in 25 grams of ethanol first. 0.1 grams of sodium EDTA (stabilizer), 1.5 grams of monoammonium glycyrrhizin (MagnaSweet 100) (sweetener), 0.02 grams of methylparaben/propylparaben 4:1 mix (Nipagin M/Nipasol M) (preservative), 0.005 grams of FD&C Red 40, 0.001 grams of Blue 1 and 0.005 grams of Yellow 5 (coloring agents) were completely dissolved in 62.47 grams of water. 8 grams of hydroxypropyl methylcellulose (Methocel E50) (water-soluble film former) was wetted and uniformly mixed with the Gelucire/ethanol solution (wetting agent), 0.5 grams of butterscotch (flavor), 1 gram of propylene glycol (plasticizer), and 0.5 grams of peppermint oil (flavor). Then the aqueous solution was gradually poured into the wetted Methocel E50 under agitation. After a homogenous viscous solution was obtained, 0.8 grams of nicotine base was added into and mixed with the solution in a well-vented environment. The final coating solution was degassed, cast dried and die-cut into unit doses. The unit dose is shown in FIG. 1.

### Example 6

### Intraoral Monolayer Film Containing Neutral and Ionized Nicotine

0.1 grams of lauroyl macrogol-32 glycerides (Gelucire 44/14) (release modifier) was dissolved in 25 grams of ethanol first. 0.1 grams of sodium EDTA (stabilizer), 1.5 grams of monoammonium glycyrrhizin (MagnaSweet 100) (sweetener), 0.02 grams of methylparaben/propylparaben 4:1 mix (Nipagin M/Nipasol M) (preservative), 0.005 grams of FD&C Red 40, 0.001 grams of Blue 1 and 0.005 grams of Yellow 5 (coloring agents) were completely dissolved in 62.47 grams of water. 7 grams of hydroxypropyl methylcellulose (Methocel E50) (water-soluble film former) and 1 grain of polyacrylic polymer (Carbopol 971P NF) (anionic mucoadhesive polymer) were wetted and uniformly mixed with the Gelucire/ethanol solution (wetting agent), 0.5 grams of butterscotch (flavor), 1 gram of propylene glycol (plasticizer), and 0.5 grams of peppermint oil (flavor). Then the aqueous solution was gradually poured into the wetted Methocel E50/Carbopol 971P NF under agitation. After a homogenous viscous solution was obtained, 0.8 grams of nicotine base was added into and mixed with the solution in a well-vented environment. The final coating solution was degassed, cast, dried and die-cut into unit doses. The unit dose is shown in FIG. 1.

The present invention having been disclosed in connection with the foregoing embodiments, additional embodiments will now be apparent to persons skilled in the art. The present invention is not intended to be limited to the embodiments specifically mentioned, and accordingly reference should be made to the appended claims rather than the foregoing discussion, to assess the spirit and scope of the present invention in which exclusive rights are claimed.

We claim:

1. A mucoadhesive film consisting essentially of
an effective dose of nicotine and
at least one non-microbial hydrocolloid,
wherein
a) the nicotine is present in a single layer;
b) wherein the non-microbial hydrocolloid has a hydration rate at 25° C. and 75% relative humidity of 5 to 20% per 24 hours;
c) the mucoadhesive film provides a nicotine peak plasma level within 15 minutes of administration to a subject; and
d) the mucoadhesive film disintegrates within 1 to 600 seconds upon application in the oral cavity or dissolves within 0.5 to 60 minutes and wherein, the non-microbial hydrocolloid is a hydroxypropyl methylcellulose having a methoxy content in the range of 19 to 30%, hydroxypropyl content in the range of 7 to 12% and molecular weight of approximately 50,000 to 250,000 daltons.

2. The mucoadhesive film of claim 1, wherein the mucoadhesive film is a monolayer intraoral film.

3. The mucoadhesive film of claim 1, wherein the mucoadhesive film is a bilayer intraoral film.

4. The mucoadhesive film of claim 2, wherein the nicotine in the mucoadhesive film is in neutral form.

5. The mucoadhesive film of claim 3, wherein the nicotine in the mucoadhesive film is in ionized form.

6. The mucoadhesive film of claim 4, wherein at least 50% of the nicotine in the mucoadhesive film is in neutral form.

7. The mucoadhesive film of claim 5, wherein at least 90% of the nicotine in the mucoadhesive film is in ionized form.

8. The mucoadhesive film of claim 1, wherein the mucoadhesive film has a water content in the range of 0.5 to 10%.

9. The mucoadhesive film of claim 3, wherein the film has a disintegration time in the range of 1 to 300 seconds.

10. The mucoadhesive film of claim 1, wherein the film has a dissolving time in the range of 0.5 to 5 minutes.

11. The mucoadhesive film of claim 1, wherein the dosage form further comprises at least one of an emulsifier, a release modifier, a taste modifying agent, a plasticizer, a water soluble inert filler, a preservative, a buffering agent, a stabilizer and a coloring agent.

12. A method of administering nicotine to a subject comprising, administering to a subject a mucoadhesive film according to claim 1, wherein the subject experiences a peak nicotine plasma level within 15 minutes of administration of the mucoadhesive film.

13. A bilayer mucoadhesive film consisting essentially of
a first layer having an effective dose of nicotine and at least one non-microbial hydrocolloid; and
a nicotine-free second layer having an alkalizing agent and at least one non-microbial hydrocolloid
wherein
a) the non-microbial hydrocolloid in each layer has a hydration rate at 25° C. and 75% relative humidity of 5 to 20% per 24 hours;
b) the mucoadhesive film provides a nicotine peak plasma level within 15 minutes of administration to a subject; and
c) the mucoadhesive film disintegrates within 1 to 600 seconds upon application in the oral cavity or dissolves within 0.5 to 60 minutes, and wherein the non-microbial hydrocolloid is a hydroxypropyl methylcellulose having a methoxy content in the range of 19 to 30%, hydroxypropyl content in the range of 7 to 12% and molecular weight of approximately 50,000 to 250,000 daltons.

14. The mucoadhesive film of claim 13, wherein the mucoadhesive film releases at least 92.01±4.53% of the original nicotine dose within 60 seconds of administration to the subject.

15. The mucoadhesive film of claim 13, wherein at least 90% of the nicotine in the mucoadhesive film is in ionized form.

16. The mucoadhesive film of claim 13, wherein the alkalizing agent is sodium bicarbonate.

17. A bilayer mucoadhesive film consisting essentially of
a) a first layer containing an effective dose of nicotine and a non-microbial hydrocolloid; and
b) a nicotine free second layer containing an alkalizing agent and a non-microbial hydrocolloid;
wherein the non-microbial hydrocolloid in each layer has a hydration rate at 25° C. and 75% relative humidity of 5 to 20% per 24 hours; and the mucoadhesive film releases at least 92.01±4.53% of the original nicotine dose within 60 seconds of administration to the subject.
